(19) United States
(12) Patent Application Publication
(10) Pub. No.: US 2011/0300641 A1
(43) Pub. Date: Dec. 8, 2011

(54) PROTEIN ISOFORMS FOR DIAGNOSIS

(75) Inventors: John J. Kopchick, Athens, OH (US); Shigeru Okada, Athens, OH (US); Sudha Sankaran, Athens, OH (US)

(73) Assignee: OHIO UNIVERSITY, Athens, OH (US)

(21) Appl. No.: 12/934,807

(22) PCT Filed: Mar. 27, 2009
(86) PCT No.: PCT/US2009/038565

§ 371 Date: Jul. 6, 2011

Related U.S. Application Data
(60) Provisional application No. 61/040,401, filed on Mar. 28, 2008.

(51) Int. Cl. G01N 33/566; C07K 14/775; H01J 49/26; C07K 16/18; G01N 27/26; G01N 33/559; C07K 14/47; C07K 1/16

(52) U.S. Cl. 436/501; 530/350; 530/359; 530/380; 530/412; 530/389.1; 530/389.3; 530/388.2; 530/388.25; 204/456; 250/282

(57) ABSTRACT

Several aspects of this invention relate to diagnosis of diabetic states in a mammal using protein isoforms. In some aspects, it relates to a method for determining the diabetic state of a mammal. This method can include, for example, (a) measuring the serum concentration of one or more protein isoforms, (b) analyzing the serum concentration of the one or more protein isoforms, and (c) determining the diabetic state of the mammal. Other aspects include kits used to perform the method. Further aspects are the isolated protein isoforms themselves, and their methods of isolation.

### RELATED APPLICATION

This Application is a U.S. National Phase of PCT/US2009/038565 entitled “Protein Isoforms For Diagnosis” filed Mar. 27, 2009, which claims the benefit of U.S. Provisional Application Ser. No. 61/040,401 entitled “Protein Isoforms For Diagnosis” filed Mar. 28, 2008, the disclosures of which is hereby incorporated herein by reference in its entirety.

### FIELD OF THE INVENTION

The present invention generally relates to methods and kits for diagnosing a disease or disorder using protein isoforms and more particularly for diagnosing the diabetic state in a mammal.

### BACKGROUND OF THE INVENTION

Several embodiments of this invention relate to diagnosis of the diabetic states in a mammal using protein isoforms, kits used to perform the method, and the isolated protein isoforms themselves and methods of isolation of the protein isoforms.

There are several types of diabetes including, for example, gestational diabetes, type 1 diabetes and type 2 diabetes; the latter being the most common form of diabetes. Diabetes can be diagnoses using a fasting blood glucose test. However, this and other tests may not provide an adequate diagnosis in that, for example, intermediate states prior to the diabetic state may not be effectively diagnosed.

For example, Yang et al. (Nature Vol. 436, pp. 356-62 (Jul. 21, 2005)) disclose that serum RBP4 levels are elevated in insulin resistant mice and humans with obesity and type 2 diabetes. Although this and other proteins have been associated with diabetes, the relative levels of protein isoforms including post-translational modified forms or splice variants may not have been evaluated for diabetes diagnosis or the intermediates states prior to the diabetic state.

### SUMMARY OF THE INVENTION

According to some embodiments of the present invention, a method is described for determining the diabetic state of a mammal comprising measuring the serum concentration of one or more protein isoforms, analyzing the serum concentration of the one or more protein isoforms, and determining the diabetic state of the mammal.

In accordance with other embodiments of the present invention, an isolated protein isoform selected from Kinninogen isoforms, Apolipoprotein A1 isoforms, Retinol Binding Protein 4 isoforms, Haptoglobin isoforms, and Transthyretin isoforms, is described. In other embodiments the method for isolating these protein isoforms is described.

Still other embodiments of the invention include a kit for the diagnosis of a state of diabetes (including pre-diabetes) in a mammal comprising a composition used for the detection of an isoform selected from Kininogen isoforms, Apolipoprotein A1 isoforms, Retinol Binding Protein 4 isoforms, Haptoglobin isoforms, and Transthyretin isoforms.

Other objects and embodiments of the present invention will be apparent in light of the description of the invention.

### BRIEF DESCRIPTION OF THE DRAWINGS

The following detailed description of specific embodiments of the present invention can be better understood when read in conjunction with the following drawings.

FIG. 1 shows the average mouse weight as a function of time for high-fat mice compared to control mice.

FIG. 2A shows average plasma insulin levels as a function of time for high-fat mice compared to control mice.

FIG. 2B-2D shows the effect of high fat diet on glucose levels, insulin levels, and intraperitoneal (IP) glucose tolerance.

FIG. 2E shows that effect of high fat feeding on glucose tolerance.

FIGS. 3A and 3B show the linear detection ranges for SYPRO Orange gel strain. Proteins represented: (FIG. 3A) β-galactosidase (◯), lysozyme (▪), bovine serum albumin (BSA, □), and phosphorylase B (); (FIG. 3B) myosin (◯), soybean trypsin inhibitor (▪), ovalbumin (□), and carbonic anhydrase ().

FIG. 4 shows a 2-D gel image of proteins and respective isoforms in mouse serum.

FIGS. 5A-5F show the 2-D gel image (A) and the total (B) and isoform (C—F) concentrations of Retinol Binding Protein 4 as a function of time for high-fat mice and control mice.

FIGS. 6A-6H show the 2-D gel image (A) and the total (B) and isoform (C—H) concentrations of Apolipoprotein A1 as a function of time for high-fat mice and control mice.

FIGS. 7A-7D show the 2-D gel image (A) and the total (B) and isoform (C-D) concentrations of Kininogen as a function of time for high-fat mice and control mice.

FIGS. 8A-8H show the 2-D gel image (A) and the total (B) and isoform (C—H) concentrations of Transthyretin as a function of time for high-fat mice and control mice.

FIG. 9 shows a 2-D gel image of proteins and isoforms in human serum. Spots marked “unknown” indicate protein isoforms that may be useful in the diagnostic method, but whose protein or isoform identify may not have been determined.

FIGS. 10A-10B show a 2-D gel image of proteins and respective isoforms in mouse serum prepared in the same way as FIG. 4 but stained with a phophoprotein stain.

### DETAILED DESCRIPTION OF THE INVENTION

The onset of type 2 diabetes (non-insulin dependent diabetes mellitus) or gestational diabetes can have several intermediate states as the mammal progresses from a normal state to a diabetic state. This progression of diabetic-related states for type 2 diabetes can proceed as follows: (1) the normal state, (2) the pre-diabetes state (also referred to as the impaired glucose tolerance state or the impaired fasting glucose state), (3) the insulin resistant/hyper-insulinemic state (e.g., that associated with obesity), and (4) the diabetic state (e.g., frank diabetes). Not all of these states necessarily occur in some progressions of diabetes. For example, progression of diabetic-related states for type 2 diabetes can proceed as follows: (1) the normal state, (2) the insulin resistant/hyper-insulinemic state (e.g., that associated with obesity), and (3) the diabetic state (e.g., frank diabetes). The development of diabetes states of the C57BL/6J mouse parallels the progression of diabetes (e.g., the progression of diabetes via the obese insulin resistant/hyper-insulinemic state) in humans.

Exemplary embodiments of the present invention include methods used to diagnose diabetic-related states. In some embodiments, diagnosis of a diabetic state includes the diagnosis of one or more of the pre-diabetes state, the insulin resistant/hyper-insulinemic state, or the diabetic state.

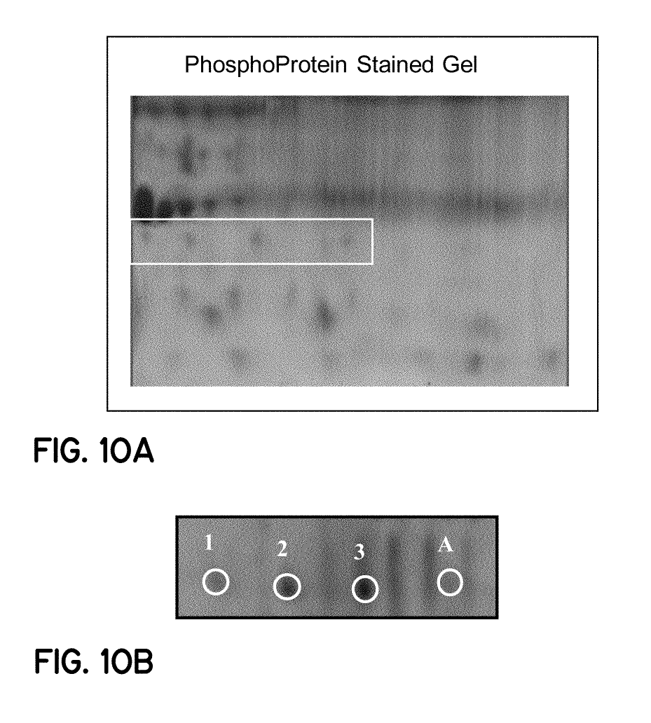

In some embodiments of the present invention, the diagnosis of a diabetic state can occur by monitoring one or more protein isoforms in plasma or serum. Protein isoforms are defined as proteins having the same amino acid sequence (or in some instances having only a few amino acid differences) but which have a different amount, type, or placement of post-translation modifications. Post-translation modifications can include, for example, phosphorylation (e.g., on tyrosine, threonine, or serine residues), glycosylation (such as O-linked or N-linked sugar groups), acylation, disulfide bond formation, oxidation and others. Post-translation modifications can be determined by any known techniques including, for example, staining using Pro-Q Diamond Phosphorylation Gel Stain (Molecular Probes; Eugene, Oreg.), as shown in FIGS. 10A-10B.

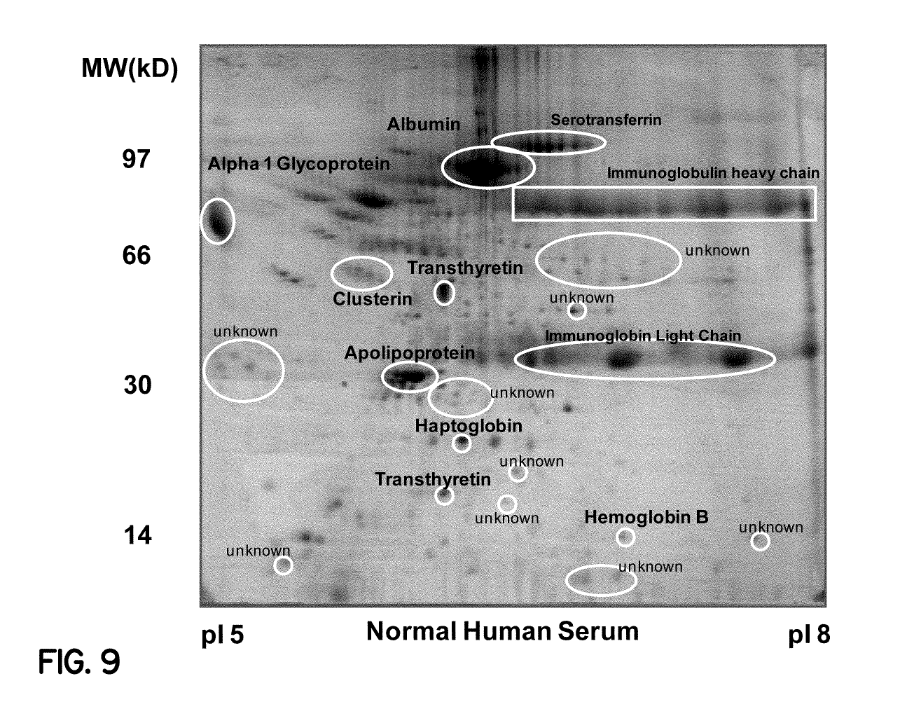

Protein isoforms that can be used to diagnose a diabetic state include any protein isoform whose plasma or serum concentration varies with diabetic state (by, for example, varying from a normal state concentration). In some embodiments, the protein isoforms can include those proteins and isoforms thereof identified in FIG. 9. In some embodiments the protein isoforms can include, but are not limited to Kininogen isoforms, Kininogen isoforms with a pl greater than about 5.5, Kininogen isoforms with a pl less than about 6.0, Kininogen isoforms having a pl of about 5.6 and about 5.7; Apolipoprotein A1 isoforms, Apolipoprotein A1 isoforms with a pl greater than about 5.0, Apolipoprotein A1 isoforms with a pl less than about 6.0, Apolipoprotein A1 isoforms with a pl of about 5.2, about 5.3, about 5.4, about 5.6, about 5.7, or about 5.8; Retinol Binding Protein 4 isoforms, Retinol Binding Protein 4 isoforms with a pl greater than about 5.0, Retinol Binding Protein 4 isoforms with a pl of less than about 6.8, Retinol Binding Protein 4 isoforms with a pl of about 5.1, about 5.5, about 5.9, and about 6.6; Transthyretin isoforms, Transthyretin isoforms with a pl greater than about 5.0, Transthyretin isoforms with a pl less than about 7.5, and Transthyretin isoforms having a pl of about 5.1, about 5.0, about 6.8, about 7.1, and about 7.3; Haptoglogin isoforms, Haptoglogin isoforms with a pl greater than about 5.5, Haptoglogin isoforms with a pl less than about 7.5, and Haptoglogin isoforms having a pl of about 5.8, about 6.2, about 6.8, and about 7.2. Some embodiments of the present invention include isolated protein isoforms or compositions that comprise substantially purified protein isoforms. Substantially purified is the isolation of the isoform from other proteins (including other isoforms) and can be at a purity of, for example, about 80%, about 90%, about 95%, or about 99% purified.

Protein isoforms concentrations can be determined or monitored using any known protein detection technique including, for example, 1D or 2D gel electrophoresis, LC (liquid chromatography), HPLC (high performance liquid chromatography), detection using monoclonal or polyclonal antibodies, absorption spectroscopy, or fluorescence spectroscopy. In some instances, 2D gel electrophoresis can be used where one dimension is a native isoelectric focusing step (which can have an error of about 0.2 pl units) and the second dimension is run under a denaturing, reducing condition to determine molecular weight (which can have an error of about 5% for MWs below about 50 kD and about 10% for MWs above about 100 kD). Other methods may include any number of HPLC techniques including, for example, ion exchange HPLC, immobilized metal ion affinity chromatography, or reversed phase separation. One or more of the above-described techniques can be coupled with a mass spectroscopic technique, such as MALDI-TOF or MS/MS. In some embodiments, samples can be collected from the gel and analyzed using mass spectrometry to determine protein identity.

To determine protein or protein isoform concentration in a gel, the gel can be labeled or stained with any label or chemical stain including, for example, radioisotopes, labeled molecules (such as radiolabeled antibodies or fluorescent-tagged molecules) or fluorescent dyes (such as SYPRO Orange or Pro-Q Diamond Phosphoprotein Gel Stain (Molecular Probes, Eugene, Oreg.)). In some embodiments, protein- or isoform-specific monoclonal or polyclonal antibodies can be used to determine or quantitate isoform or protein concentration. For example, Western blots or Elisa assays can be used to determine or quantitate isoform or protein concentration.

Analysis of one or more protein isoform concentrations can provide a diagnosis of a diabetic state. In some embodiments, the analysis of one or more protein isoform concentrations can include analysis of one or more protein isoform concentrations of the same animal sampled at one or more times. For example, the difference in time of sampling of the animal can be 1 day, 3 days, 5 days, a week, two weeks, four weeks, two months, four months, six months, one year, two years, or longer periods of time (e.g., if a change is sought to be determined from a baseline measurement made five or more years prior to a change in diabetic state). Of course, multiple sampling can occur (e.g., 2, 3, 5, 7, 10, 12, 20, or more samplings) and can be included in the analysis. The differences in time between three or more samplings can be the same, can be different, and can include sampling schemes where some differences in time are the same and some are different. Sampling schemes (including, for example, choices of sampling times (e.g., sampling time differences), or protein isoform concentrations to be sampled at one or more of those sampling times) can be designed as desired.

Analysis can include, for example, measuring the concentration of a single protein isoform at one or more times; measuring the concentration ratio of two protein isoforms at one or more times; or measuring the concentrations of two or more protein isoforms at one or more times to assess conformance against predicted, pre-determined, or expected trends (e.g., such as a predetermined baseline). In some embodiments, this analysis can include concentration measurement(s) for a single time point or can include two or more such measurement(s) at different times. In other embodiments, the analysis comprises a measurement that can be made when the mammal is in a normal state and then a later-in-time measurement can be compared to the normal-state measurement. Other embodiments include analyses that use a single time measurement that is then compared against a population average or other norm-based determination (e.g., for all mammals or a species of mammals or subpopulations within a given species or for that individual). The population average or other norm-based determination can be, for example, experimentally measured or interpolated or extrapolated from other data. The population average or other norm-based determination can be determined for the normal state, the pre-diabetes state, the insulin resistant/hyper-insulinemic state, or the diabetic state. In another embodiment, the population average or other norm-based determination can be a baseline to which a single-time determination can be compared.

Analysis using concentration measurements of multiple protein isoforms can be performed. For example, protein isoform analysis (as, for example described above) can be performed and then each analysis is compared individually including any variations with time. In other embodiments, analysis is performed using a multivariate analysis that takes into consideration multiple protein isoform concentrations with, for example, a single equation, mathematical model, computational model, or conceptual model. Of course, variation of isoform concentrations with time can be incorporated into the multivariate analysis. As with the other types of analyses, for example, this use of multiple protein isoform concentration can be performed on a single time point or using two or more time points, in, for example a trend analysis or comparison to a normal state.

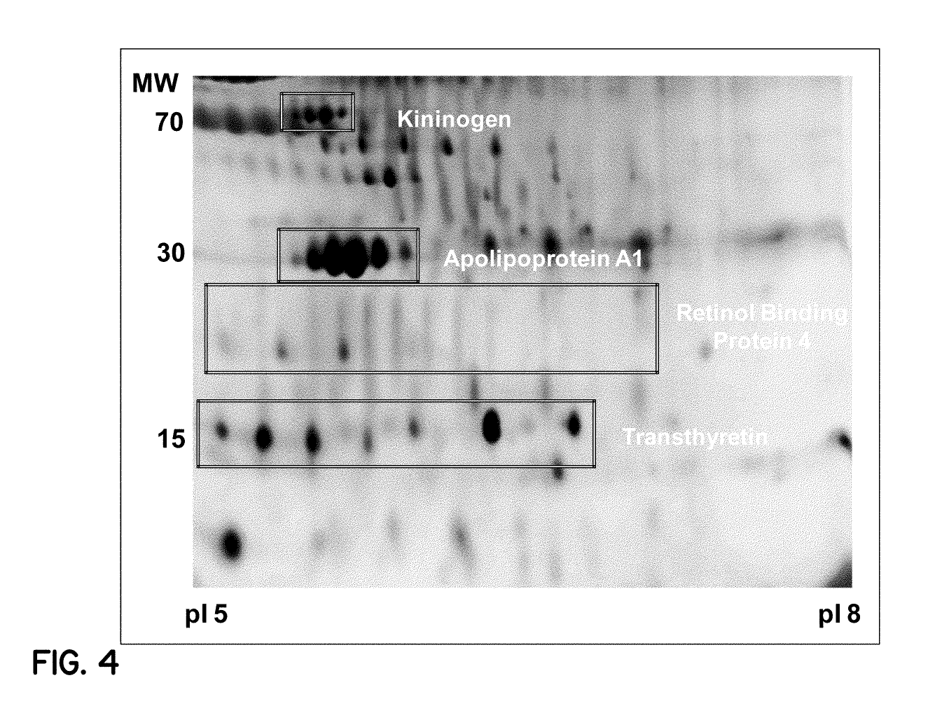

This method of diagnosis can be applied to any mammal including for example, mice, rats, humans, dogs, cats, horses, cattle, pigs, meat cattle, dairy cattle, zoo animals, farm animals, and exotic species. Protein isoforms of several of the same proteins can be found for mice and humans, as demonstrated for example by the 2-D gel images of mouse serum and human serum of FIGS. 4 and 9, respectively. Protein isoforms that may be useful in the method can be identified by examining how protein isoform concentrations vary with changes in diabetic state (e.g., by inducing the diabetic state as below or determining it by other means).

### EXAMPLES

Isoforms of proteins in mice were determined as they develop diet induced diabetes. A set of 20 diet induced type 2 diabetic mice were used to evaluate plasma proteomic changes during the progression of animals from the normal state, to the pre-diabetes state, the insulin resistant/hyper-insulinemic state, and finally to a type 2 diabetic state. C57BI/6J male mice were reared either on regular chow or a high-fat diet at weaning and their physiological responses (i.e., weight, fasting plasma glucose and insulin, and glucose tolerance) were monitored at regular time intervals. Throughout the study, plasma was collected for proteomic analysis by 2D gel electrophoresis and mass spectrometry. Protein levels were quantified by gel image analysis.

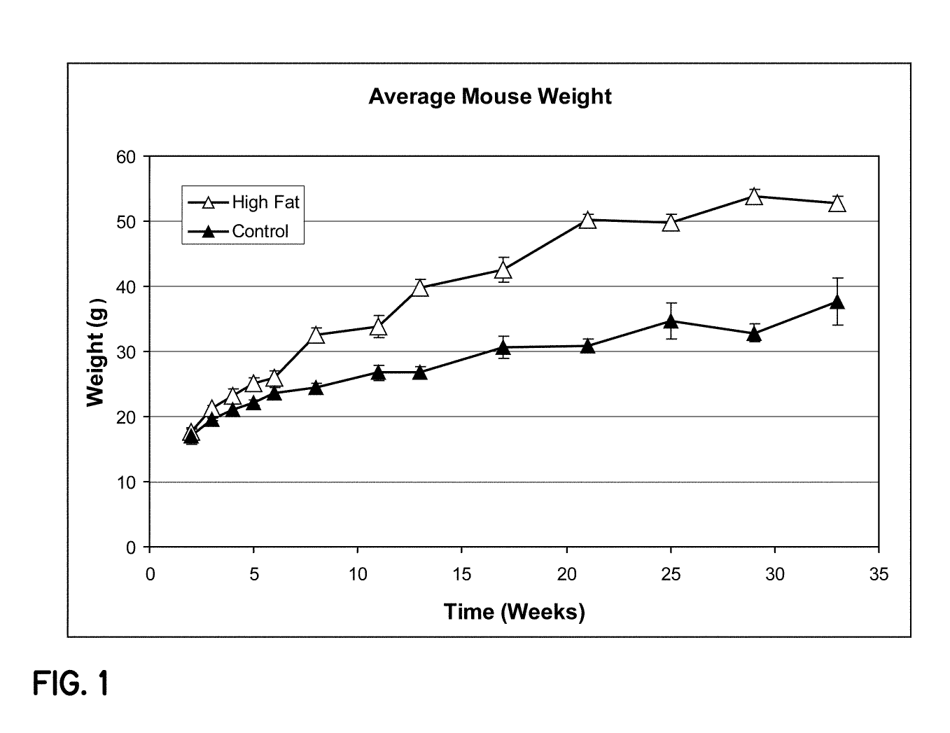

Generating obese and obese/Type 2 diabetic mouse models using a high-fat diet: Standard strains of mice C57BI/6J were used for all studies. Obese and obese/type 2 diabetic mice (referred to as high fat mice) were generated by feeding the animals a high-fat diet purchased from Bioserve (Frenchtown, N.J.) in which 17% of the calories were provided by protein, 27% of the calories were provided by carbohydrates, and 56% were provided by fat. The control diet was a standard Rodent Chow purchased from Purina (Brentwood, Mo.) in which 26% of the calories were provided by protein, 60% were provided by carbohydrates, and 14% were provided by fat. Mice were housed two per cage in a temperature-controlled room. All mice were allowed ad libitum access to water and one of the two diets. All mice were weighed weekly. FIG. 1 shows the average mouse weight as a function of time for control mice and high fat mice.

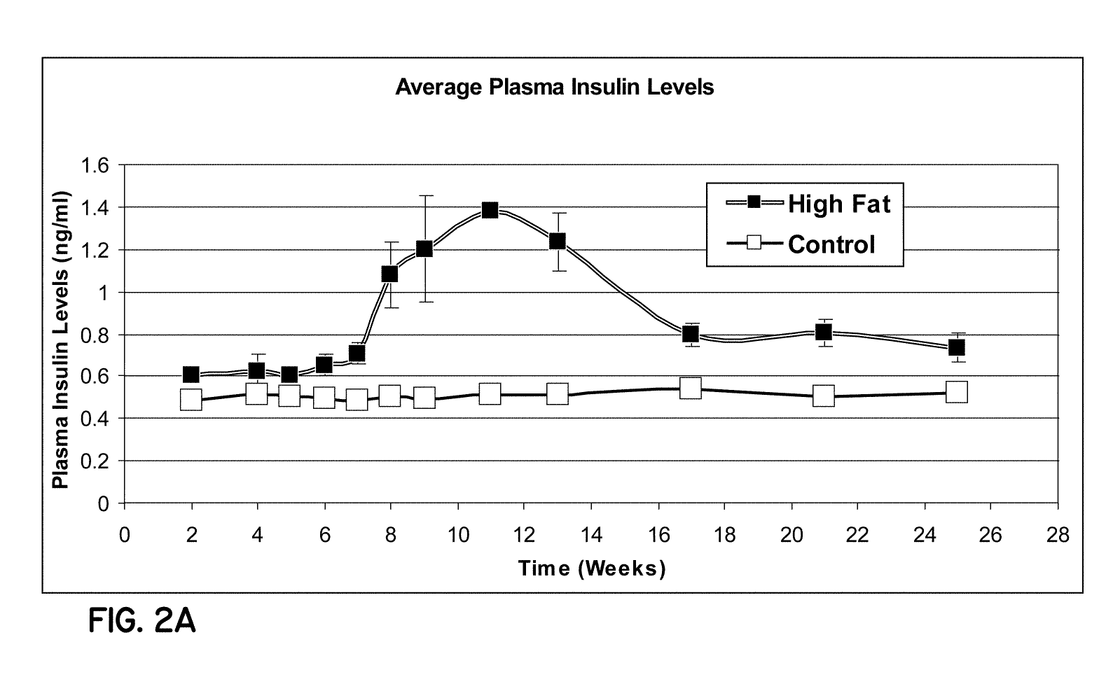

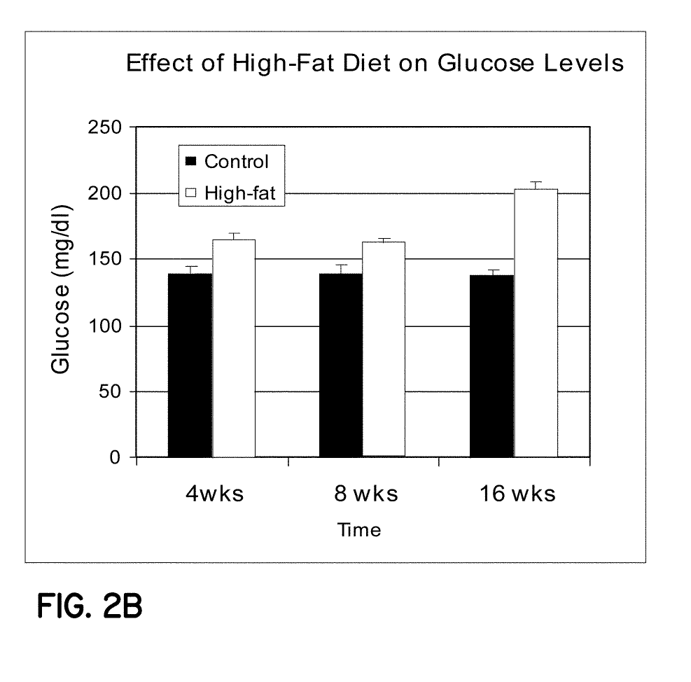

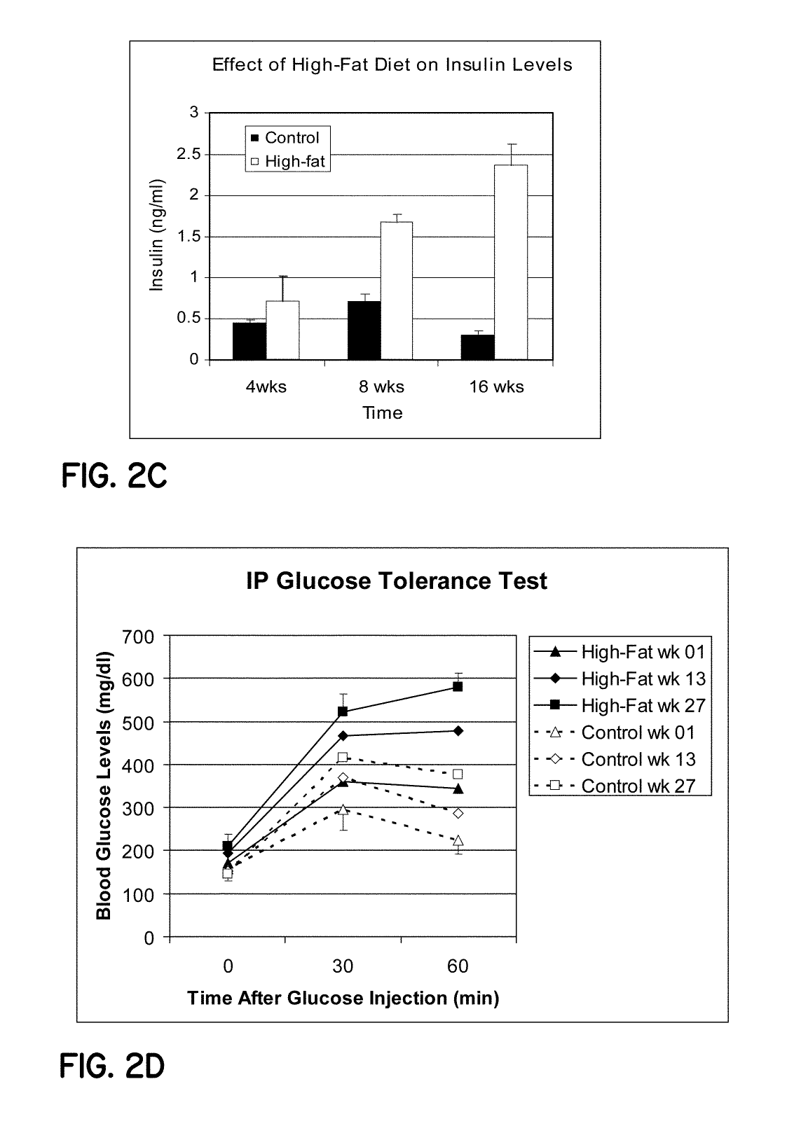

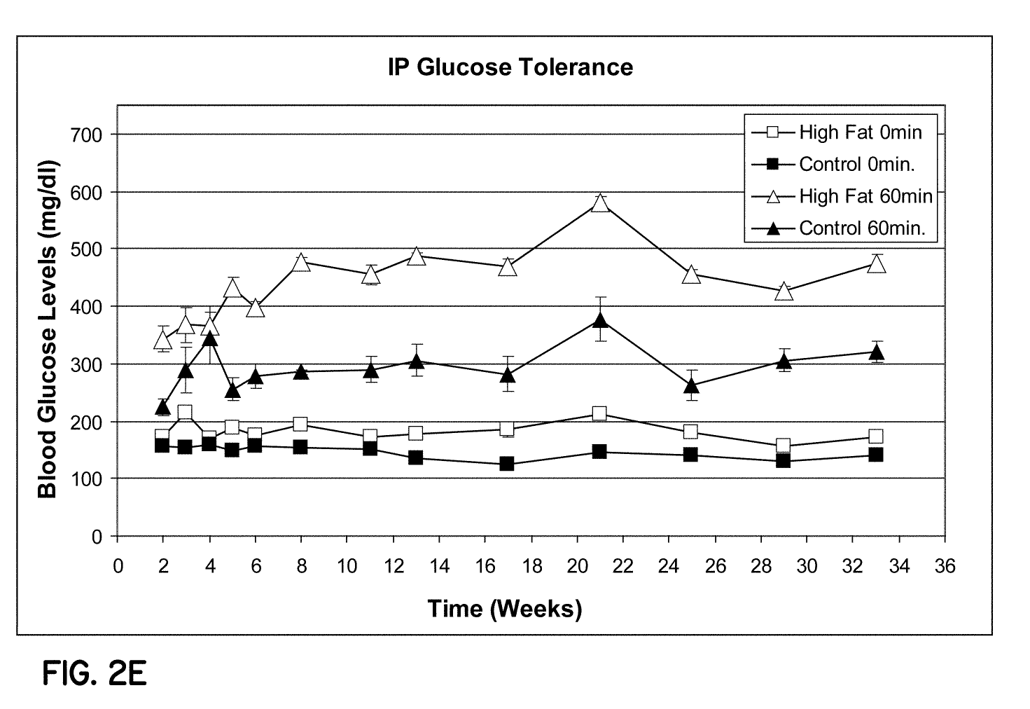

Insulin and Glucose Measurements: Four hour fasting blood samples was collected from the tail into a heparinized capillary tube and stored on ice. Plasma was separated from the cellular components by centrifugation for 10 minutes at 7000 g and then stored at −20° C. Insulin concentrations were determined using the Rat Insulin ELISA kit and rat insulin standards (ALPCO: Windham, N.H.) as per the instructions of the manufacturer. Values were adjusted by a factor of 1.23 as determined by the manufacturer to correct for the species differences in cross-reactivity with the antibody. FIG. 2A shows average plasma insulin levels after four hours of fasting as a function of time for control and high fat mice. The plotted results are averages (n=15 high-fat diet, n=5 controls) up to 25 weeks after high-fat feeding.

FIGS. 2B-2D shows the effect of high fat diet on glucose levels, insulin levels, and intraperitoneal (IP) glucose tolerance. These data were generated using the procedures described above, with the following two changes. First, ten control mice and fifty high fat mice were used. Second, two insulin resistant/hyper-insulinemic and four diabetic mice from the high fat group were sacrificed at each of 2, 4, 8, and 16 weeks. Therefore, the animals that showed early response to high fat feeding were removed from the population and are not reflected in the data of FIGS. 2B-2D.

FIG. 2E shows the effect of high fat feeding on glucose tolerance. Glucose tolerance was measured by intraperitonealy injecting a 25% glucose solution at 0.01 mL/gm body weight after four hours of fasting. Blood glucose was measured immediately before the glucose injection (0 min.) and at 30, 60, and 90 min. after the injection by tail bleeding using the ONE TOUCH glucometer from Lifescan (Milpitis, Calif.). Average blood glucose levels of high fat fed group (n=10) and control group (n=5) at 60 min. after glucose injection were plotted against time on high-fat diet. In order to double the number of blood collections while minimizing the stress of bleeding on the mice, each of control (n=10) and high-fat fed (n=20) cohort was divided into two bleeding groups and blood was collected every other week from each group until 8 weeks. Then blood collection was continued at the indicated time points by alternating between the bleeding groups.

Protein isolation: Serum samples Serum samples were collected at the respective time points by tail bleeding. Mice were sacrificed by cervical dislocation. Tissues were collected and cleaned and washed in washing buffer (Cold Saline (0.15M NaCl) with 20 ul/10 ml protease inhibitor cocktail). Nuclei and organelles were removed by low-speed spin (25,000 rpm). The supernatant was aliquoted and stored at −80° C. The protein concentration was determined by spectrophotometry.

2D-Gel Electrophoresis: Protein samples (serum or homogenized supernatants) were solubilized in sample buffer (8 M urea, 1.8 M thiourea, 4% CHAPS and carrier ampholytes) followed by reduction and alkylation using TBP (tributylphosphoine) and IAA (iodoacetamide), respectively. The samples were iso-electrically focused (IEF) using immobilized pH gradient (IPG) strips. The second dimension of the electrophoresis was performed on 15% SDS-PAGE gels under reducing conditions. Gels were fixed and then stained with the fluorescent dye SYPRO Orange, as described below.

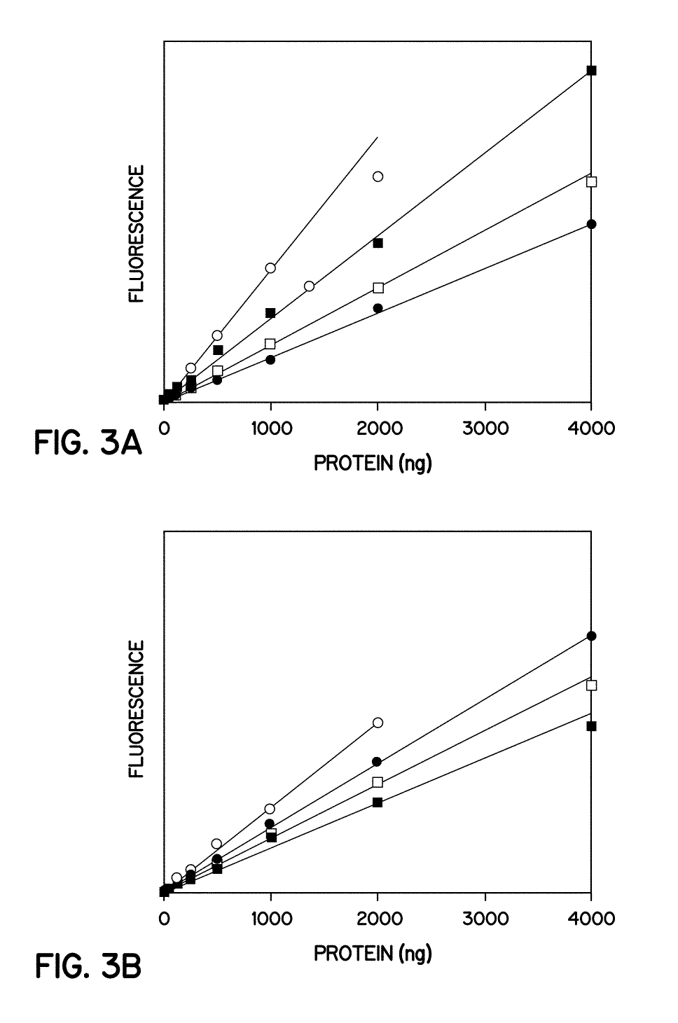

Quantitation of proteins and isoforms in a gel using SYPRO Orange protein gel stain (S6650, S6651): Detection limit and linearity of protein quantification were validated by the manufacturer as follows. A protein mixture was serially diluted and electrophoresed on a 15% SDS-polyacrylamide gel and then stained with SYPRO Orange protein gel stain. The gel was then scanned using a Molecular Dynamics Storm gel and blot analysis system (excitation/emission 488/>520 nm) and analyzed to yield the fluorescence intensities of the stained bands. The fluorescence intensity scale was the same in both panels, illustrating the small degree of protein-to-protein staining variation of the SYPRO Orange gel stain. Detection limits are between 2 and 16 ng of protein; the linear detection ranges are approximately 1000-fold. Proteins represented in FIGS. 3A and 3B are: (FIG. 3A) β-galactosidase (◯), lysozyme (▪), bovine serum albumin (BSA, □), and phosphorylase B (); (FIG. 3B) myosin (◯), soybean trypsin inhibitor (▪), ovalbumin (□), and carbonic anhydrase ().

Image capture, mass spectrometry and analysis: Images were captured with a laser-scanning device (Fuji FLA-3000G) and analyzed with PDQuest software. After correcting for loading and background, the relative density of each spot was analyzed. Proteins or isoforms of interest were manually excised from the gels and analyzed by mass spectrometry at the University of Michigan Protein Consortium. Both MALDI-TOF (Voyager-DE Pro, Applied Biosystems) and MS/MS (4700 Proteomics Analyzer, Applied Biosystems) were used to obtain probability-based peptide mass fingerprints, which were queried using the Mascot server hosted by Matrix Science (<<http://www.matrixscience.com>>) and the MSFIT server hosted by UCSF (<<http://prospector.ucsf.edu>>) to identify the spots. The excision of proteins or isoforms from the gel provides a composition comprising the isolated protein or isolated protein isoform that is substantially purified.

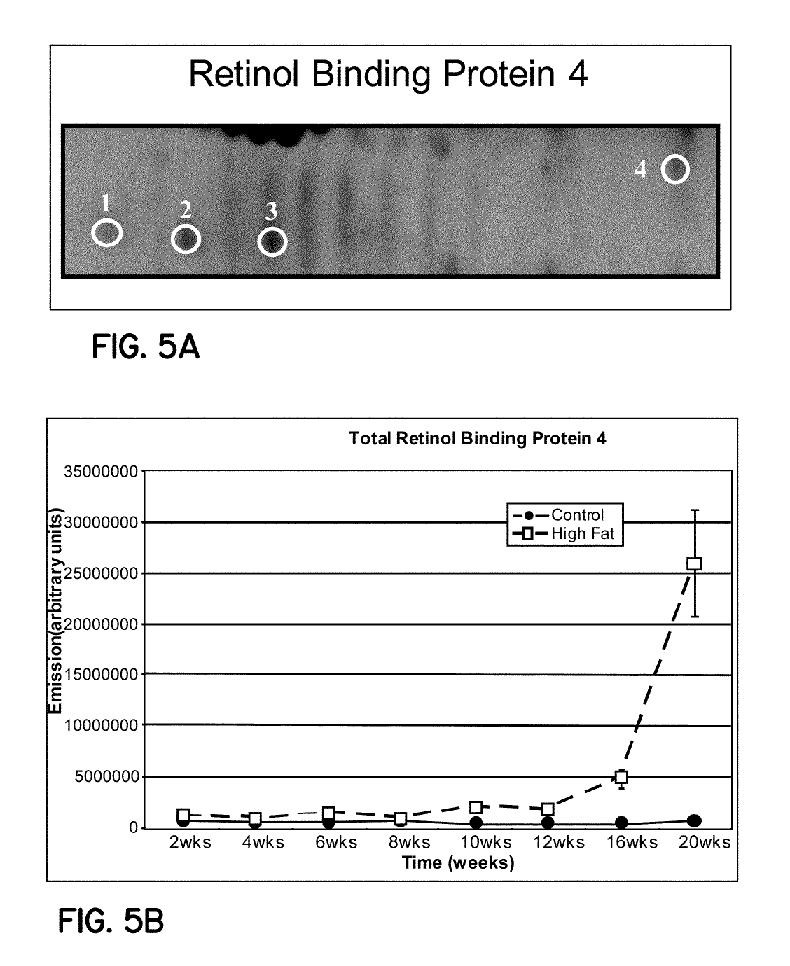

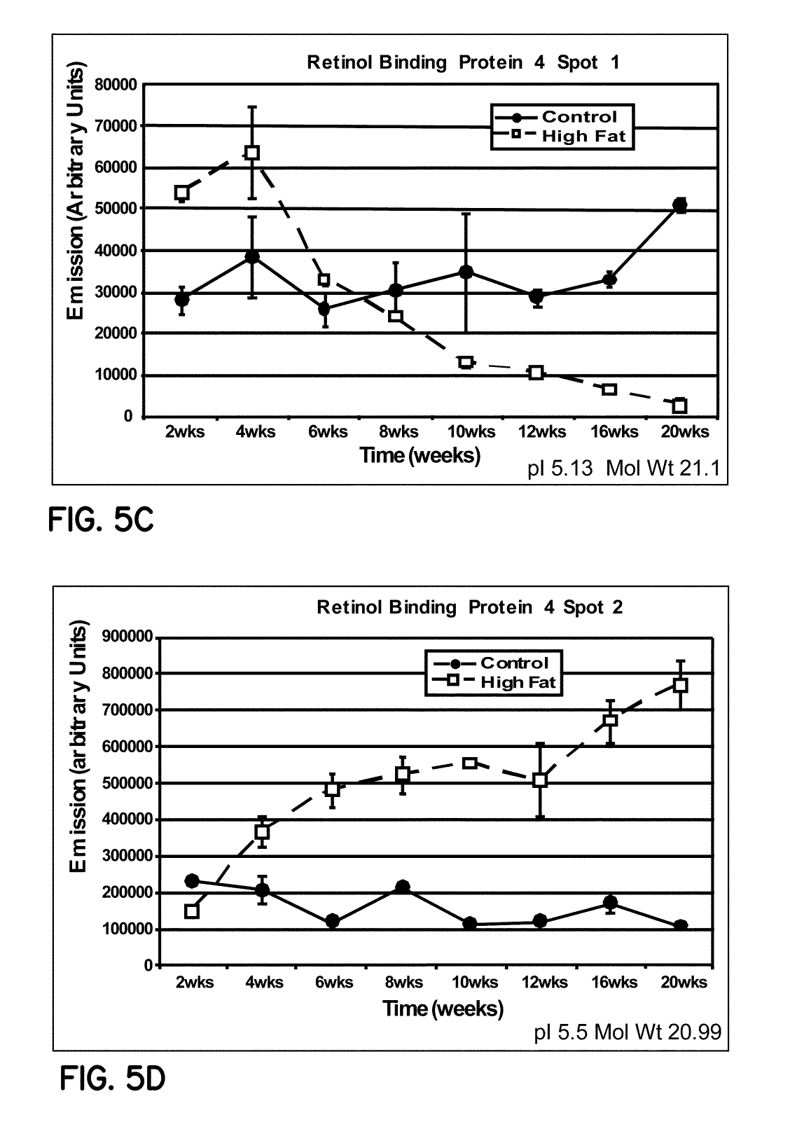

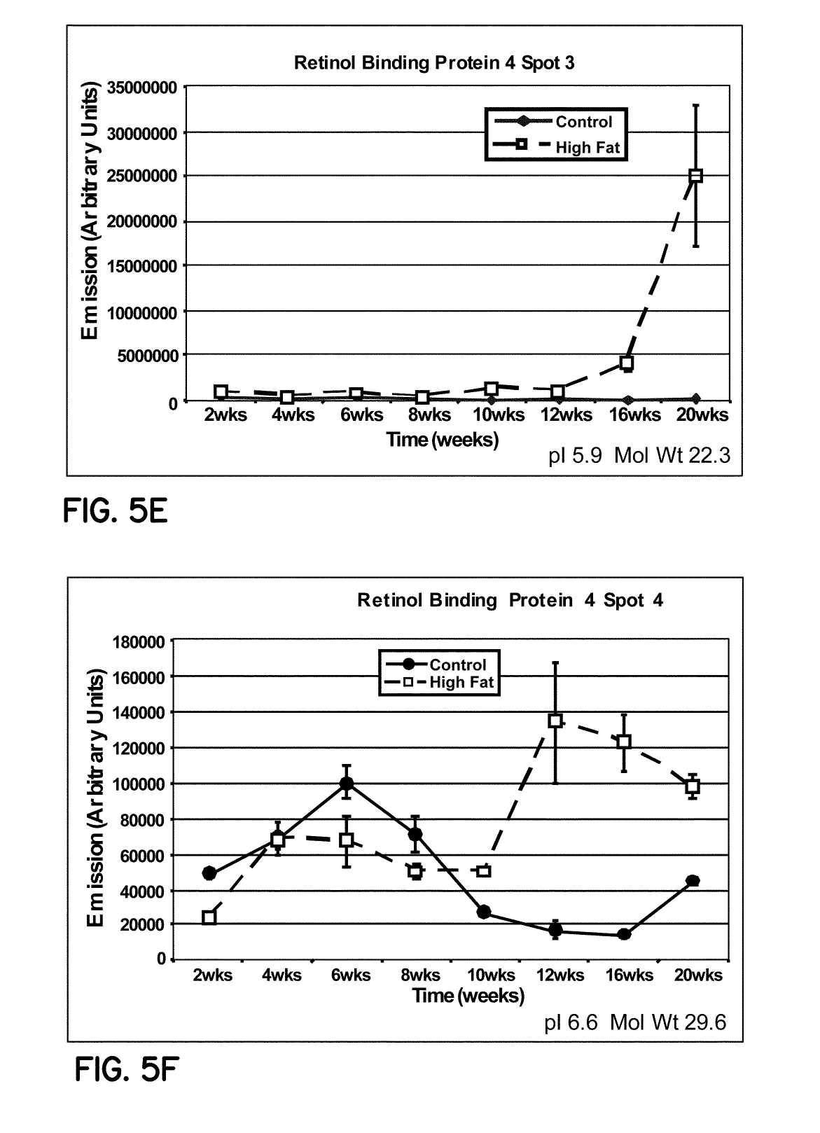

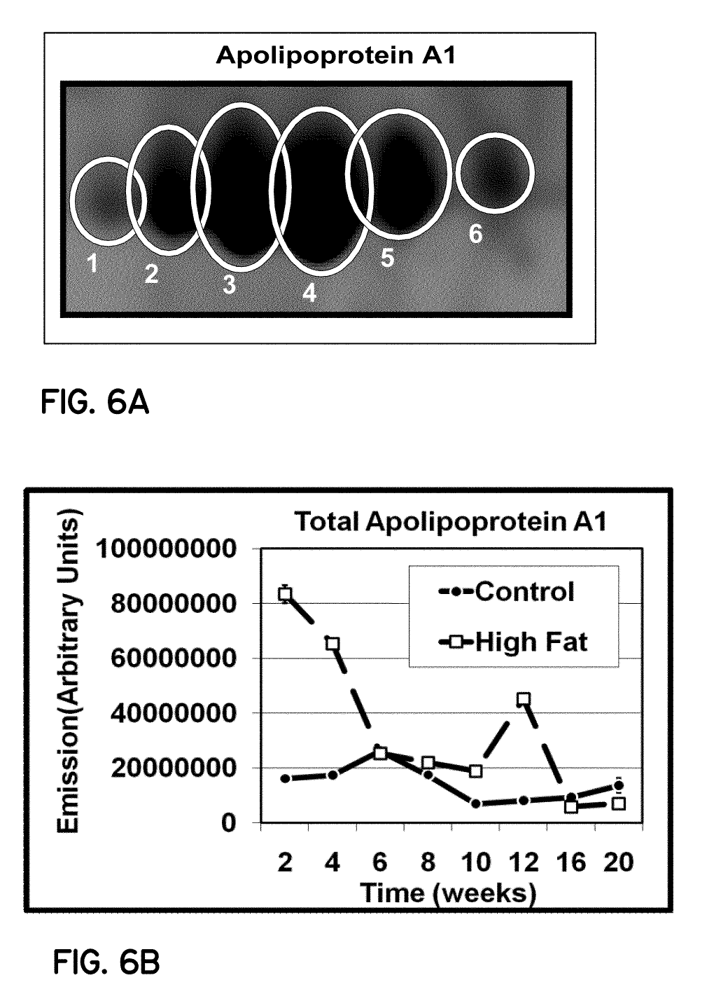

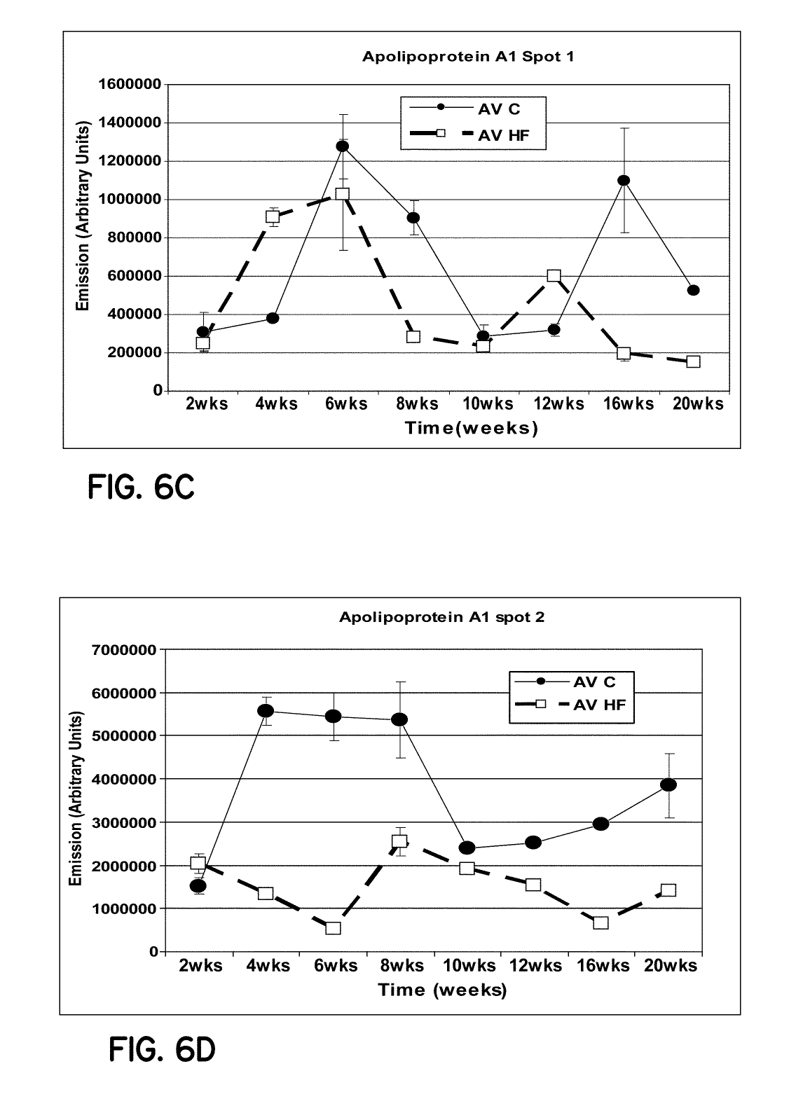

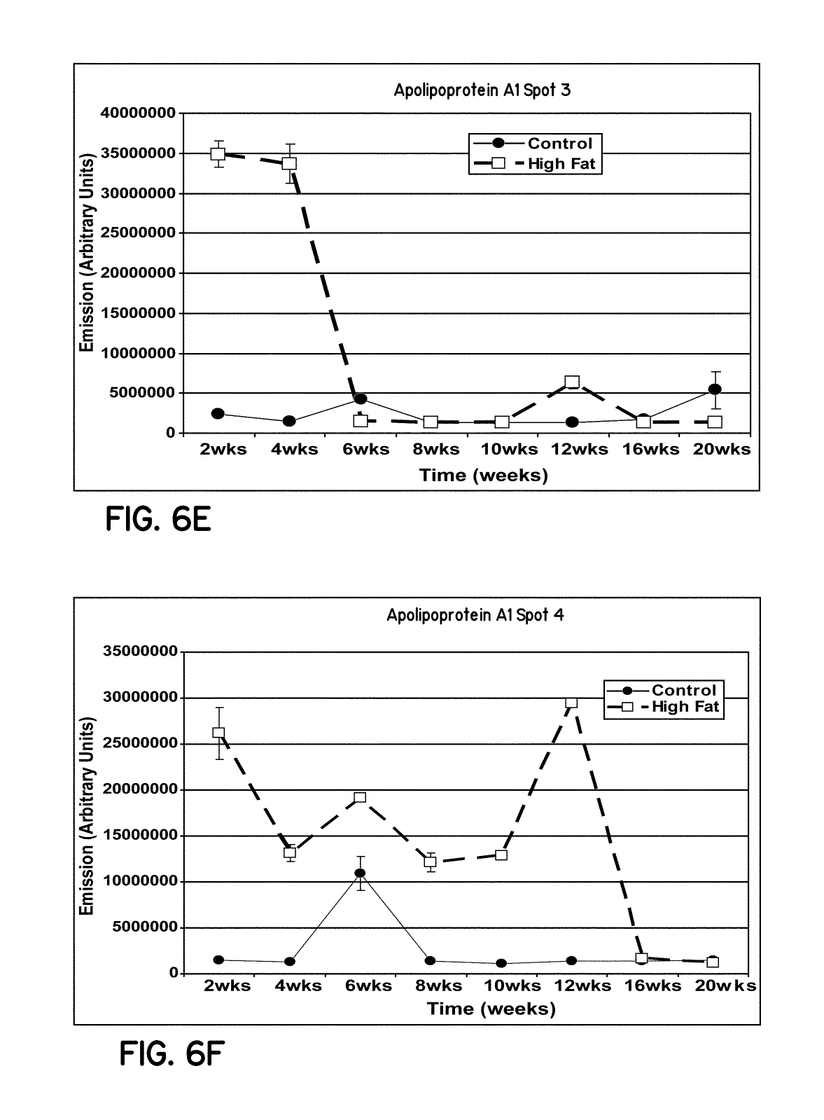

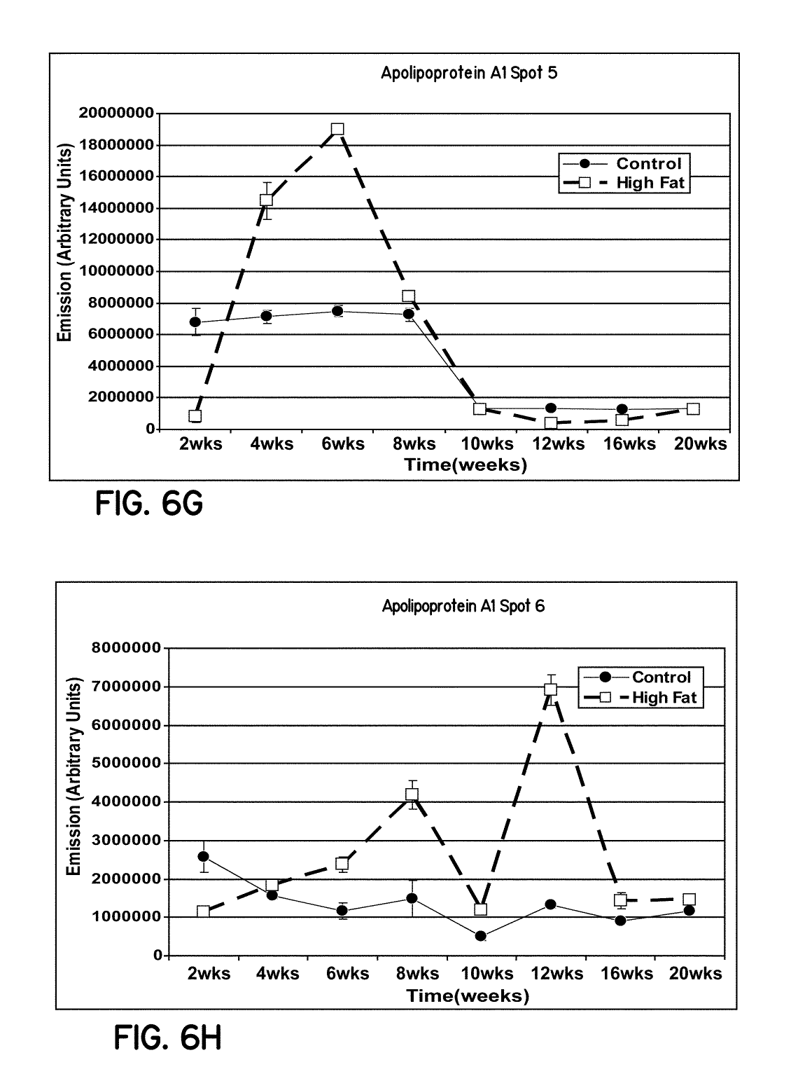

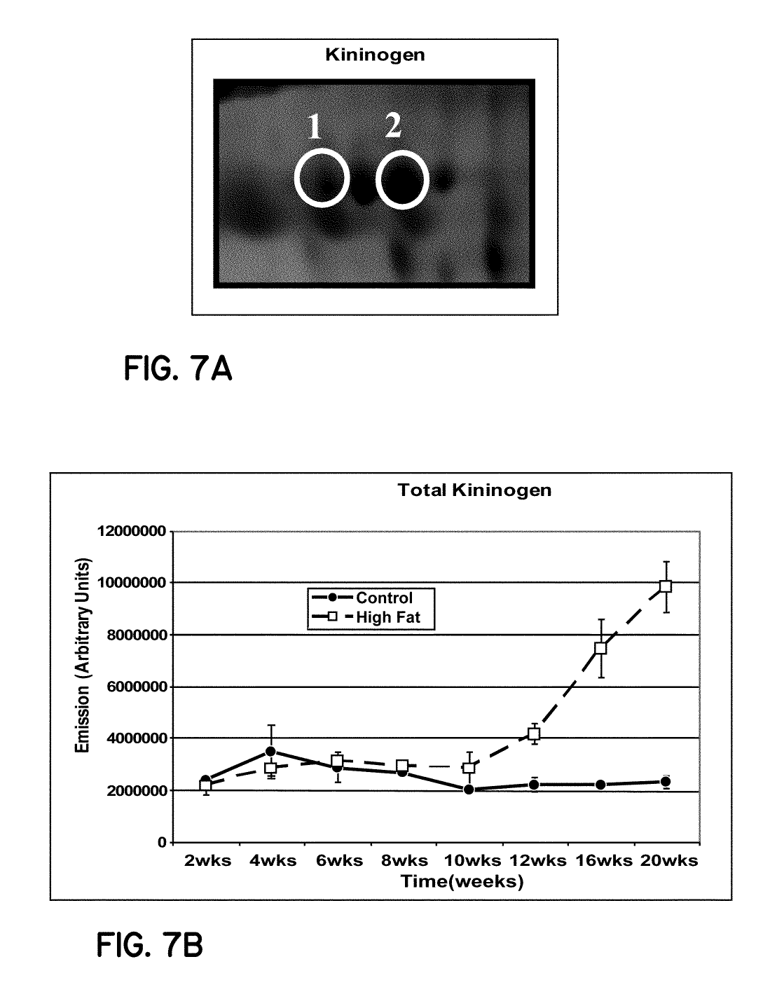

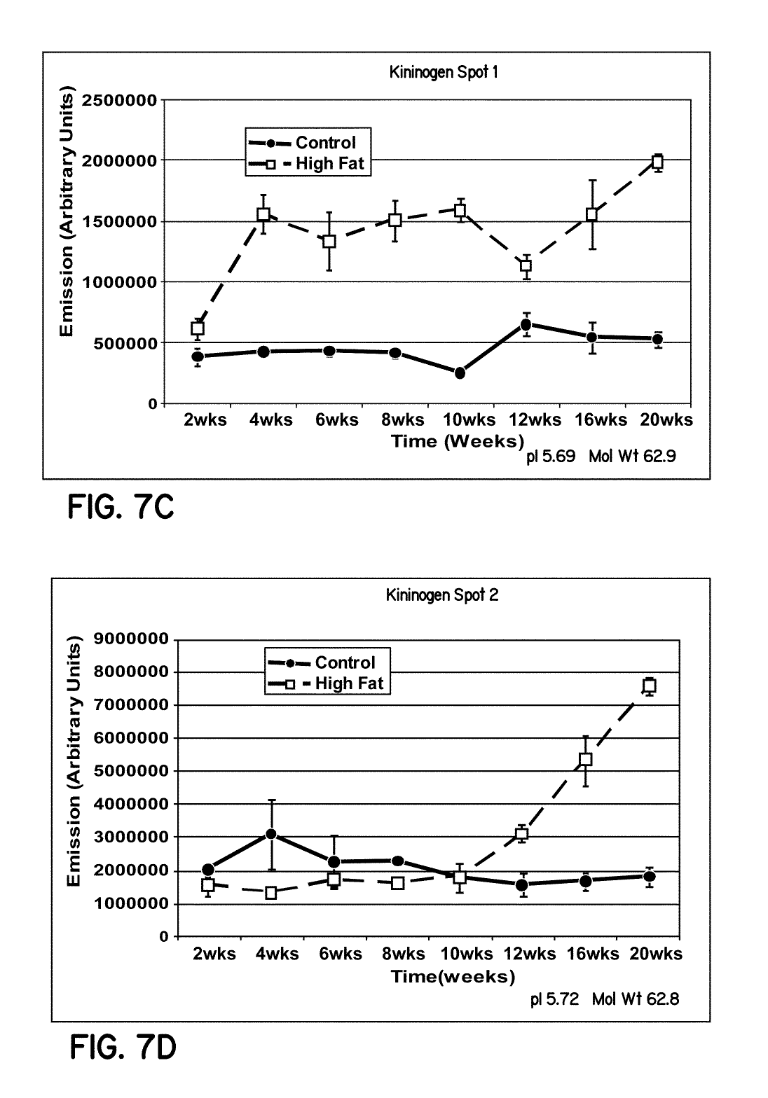

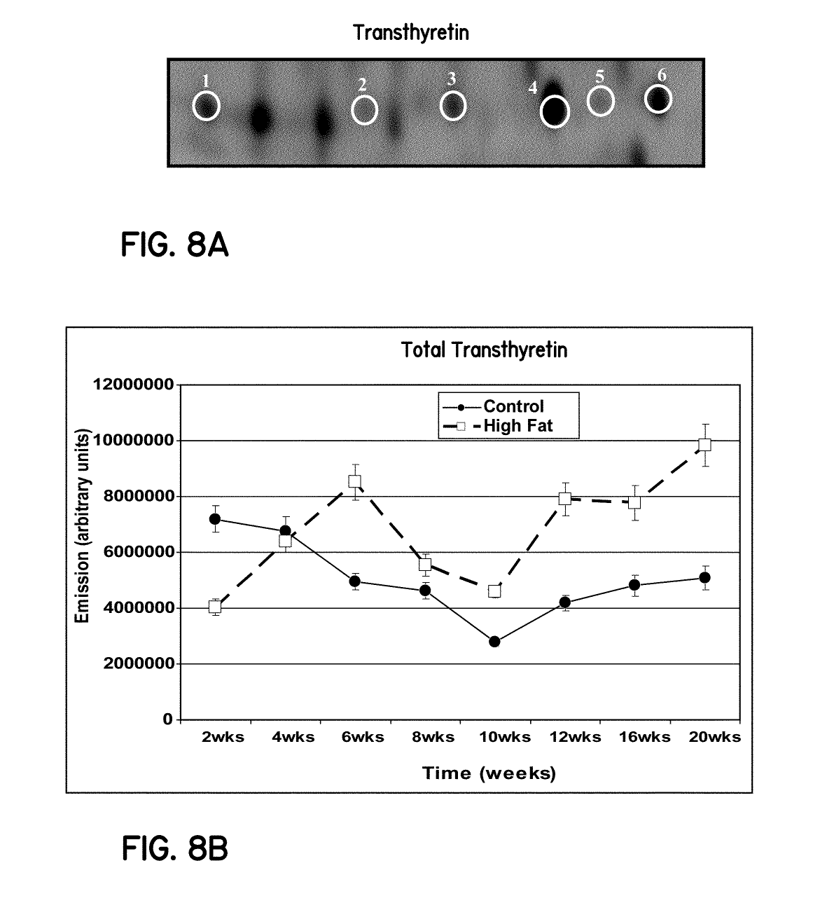

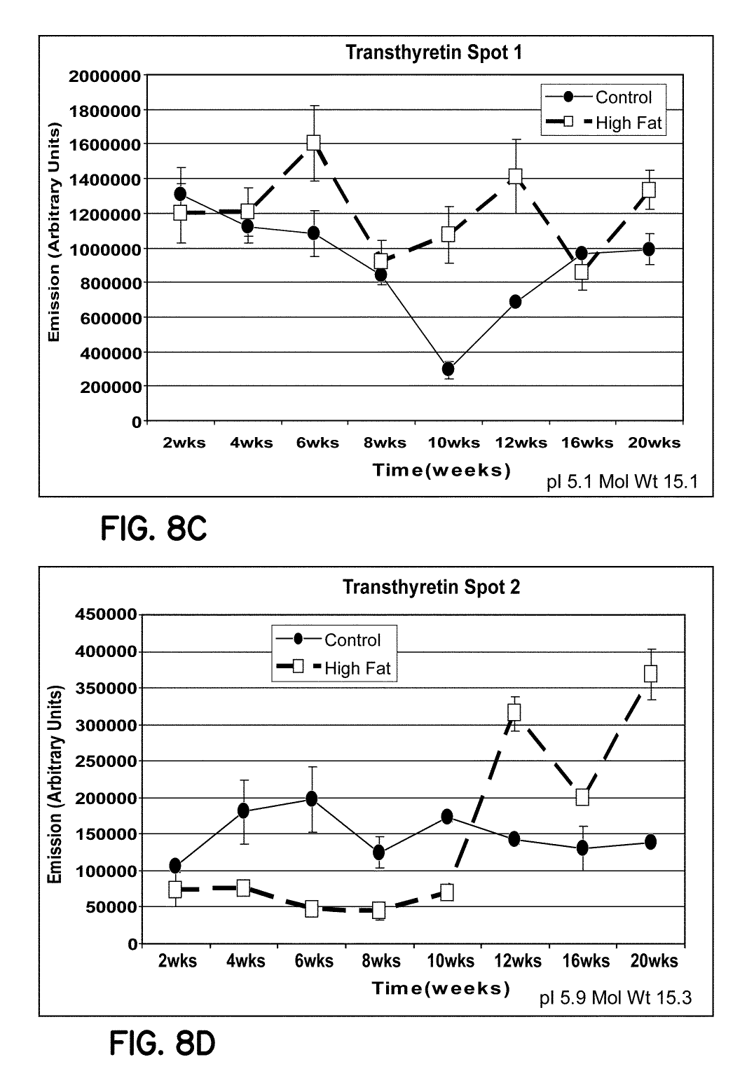

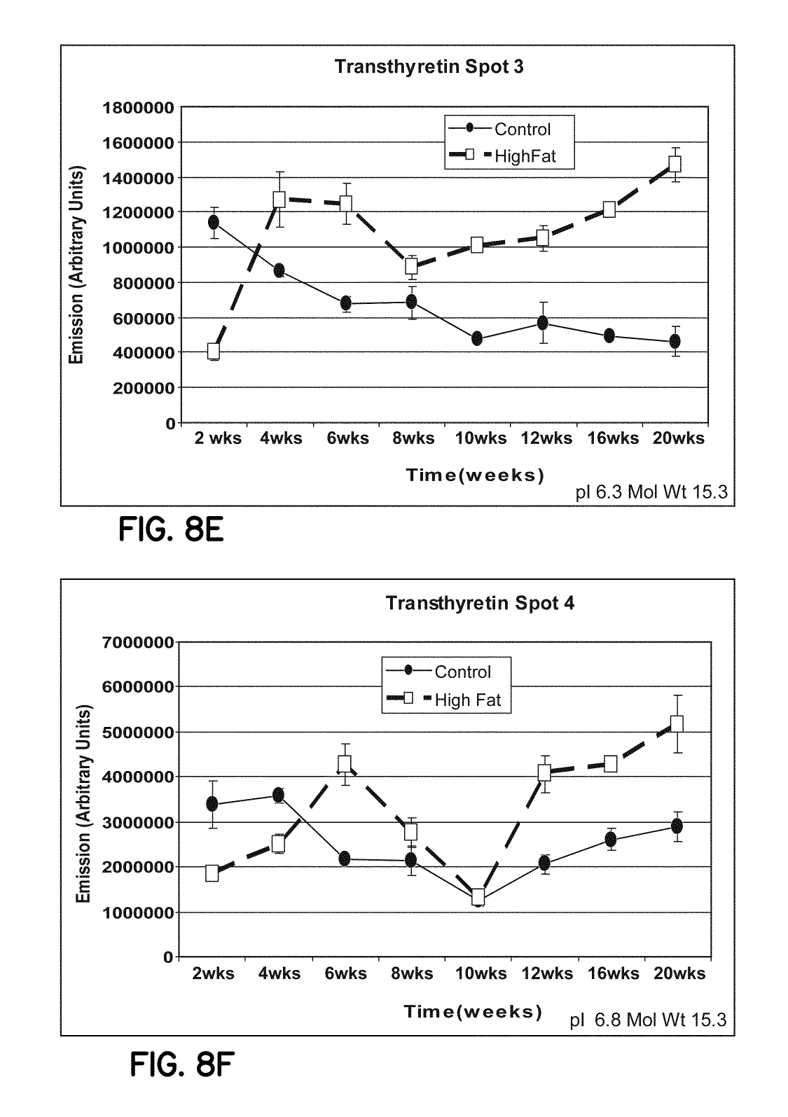

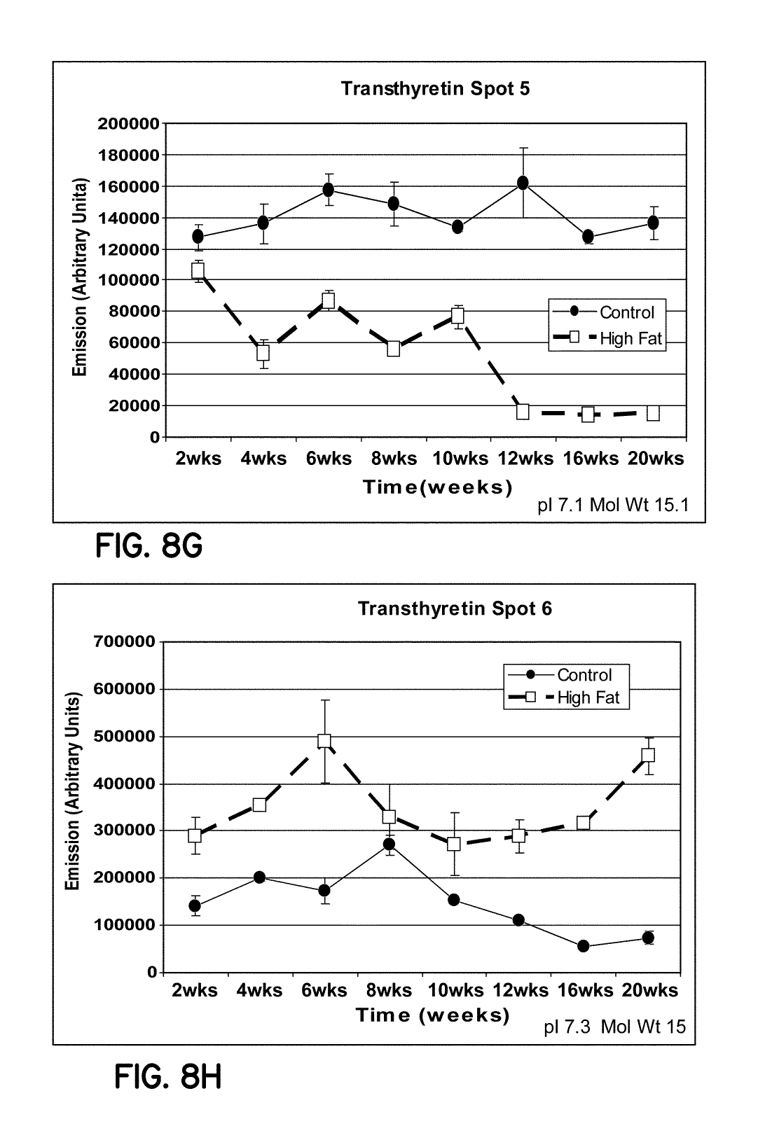

Onset of the insulin Onset of the insulin resistant/hyper-insulinemic state was observed as early as 2 weeks on a high fat diet with corresponding glucose intolerance. Fasting blood glucose levels rose significantly after 4 weeks on the high-fat diet. Nine plasma proteins showed significant changes as early as 4 weeks. Several proteins revealed multiple isoforms, some of which were extend over 2 or more isoelectic points (pl), as demonstrated by FIG. 4. For example, four spots were identified as retinol binding protein 4 (RBP4) where the molecular weights (MW) ranged from about 17 kD to about 20 kD, and the pl's were about 5.0 to about 7.0; see FIGS. 5A-5F. The total plasma concentration of RBP4 began to increase at 16 weeks and reached very high levels by 20 weeks on the high-fat diet. Control levels of this protein stayed low throughout the duration of the experiment. The most acidic form (about pl 5.1) of RBP4 was up regulated between 2 and 4 weeks after high fat feeding. The level then steadily decreased after 4 weeks. The isoform of RBP4 at about pl 5.5 steadily increased in mice on the high-fat diet. The increase in this form precedes the onset of diet-induced insulin resistant/hyper-insulinemia, thereby indicating the pre-diabetic state. The isoform of RBP4 of about pl 5.9 appeared most abundant and thus may dictate the change of total RBP4; this isoform did not start to increase until 12 weeks. Similarly, isoform regulation was found for Apolipoprotein A1 (FIGS. 6A-6H), Kininogen (FIGS. 7A-7D), and Transthyretin (FIGS. 8A-8H). These results demonstrate that this proteomic approach, especially when isoforms are monitored, can be used as a diagnostic tool to determine a normal state, a pre-diabetes state, insulin resistant/hyper-insulinemic state, and a type 2 diabetes state.

Quantitation of proteins and isoforms in a gel using Phosphoprotein gel stain: A 2-D gel image of proteins and respective isoforms in mouse serum was prepared in the same way as FIG. 4 but stained with the phophoprotein stain Pro-Q Diamond Phosphoprotein Gel Stain (Molecular Probes, Eugene, Oreg.). FIG. 10B is an exploded view of the rectangle shown in FIG. 10A. Protein spots stained for phosphorylation appeared at similar locations as RBP4 isoforms 1, 2, and 3 compared to FIG. 4. An additional spot (labeled A) only appears on the phosphoprotein stained gel.

Having described the invention in detail and by reference to some specific embodiments thereof, it will be apparent that modifications and variations are possible without departing from the scope of the invention defined in the appended claims. More specifically, although some aspects of the present invention are identified herein as preferred or particularly advantageous, it is contemplated that the present invention is not necessarily limited to these preferred aspects of the invention.

What is claimed is:

1. A method for determining a diabetic state of a mammal comprising
measuring a concentration of one or more protein isoforms in the mammal's plasma or serum,
analyzing the concentration of the one or more protein isoforms, and
determining the diabetic state of the mammal.

2. The method of claim 1 wherein the one or more protein isoforms is selected from the group consisting of: Kininogen isoforms, Apolipoprotein A1 isoforms, Retinol Binding Protein 4 isoforms, Haptoglobin isoforms, and Transthyretin isoforms.

3. The method of claim 1 wherein the one or more protein isoforms are selected from the group consisting of: Kininogen isoforms with a pl ranging from about 5.5 to about 6.0; Apolipoprotein A1 isoforms with a pl ranging from about 5.0 to about 6.0; Retinol Binding Protein 4 isoforms with a pl ranging from about 5.0 to about 6.8; and Transthyretin isoforms with a pl ranging from about 5.0 to about 7.5.

4. The method of claim 1 wherein the one or more protein isoforms are selected from the group consisting of: Kininogen isoform with a pl of about 5.6; Kininogen isoform with a pl of about 5.6; Apolipoprotein A1 isoform with a pl of about 5.2; Apolipoprotein A1 isoform with a pl of about 5.3; Apolipoprotein A1 isoform with a pl of about 5.4; Apolipoprotein A1 isoform with a pl of about 5.6; Apolipoprotein A1 isoform with a pl of about 5.7; Apolipoprotein A1 isoform with a pl of about 5.8; Retinol Binding Protein 4 isoform with a pl of about 5.1; Retinol Binding Protein 4 isoform with a pl of about 5.5; Retinol Binding Protein 4 isoform with a pl of about 5.9; Retinol Binding Protein 4 isoform with a pl of about 6.6; Transthyretin isoforms with a pl of about 5.1; Transthyretin isoforms with a pl of about 5.9; Transthyretin isoforms with a pl of about 6.3; Transthyretin isoforms with a pl of about 6.8; Transthyretin isoforms with a pl of about 7.1; and Transthyretin isoforms with a pl of about 7.3.

5. The method of claim 1 wherein the mammal is a mouse or a human.

6. The method of claim 1 wherein the diabetic state that is determined is the insulin resistant/hyper-insulinemic state.

7. The method of claim 1 wherein the diabetic state that is determined is the pre-diabetes state.

8. The method of claim 1 wherein measuring the plasma or serum concentration is accomplished using a method that comprises gel electrophoresis or antibodies.

9. The method of claim 1 wherein the analyzing comprises comparing one or more protein isoform concentrations to the concentrations of the same one or more protein isoforms performed at a time prior to the measuring.

10. The method of claim 1 wherein the analyzing comprises determining the ratio of the concentrations of two of the one or more protein isoforms.

11. An isolated protein isoform selected from the group consisting of: Kininogen isoforms, Apolipoprotein A1 isoforms, Retinol Binding Protein 4 isoforms, Haptoglobin isoforms, and Transthyretin isoforms.

12. The isolated protein isoform of claim 11 wherein the isoform is selected from the group consisting of: Kininogen isoforms with a pl ranging from about 5.5 to about 6.0; Apolipoprotein A1 isoforms with a pl ranging from about 5.0 to about 6.0; Retinol Binding Protein 4 isoforms with a pl ranging from about 5.0 to about 6.8, and Transthyretin isoforms with a pl ranging from about 5.0 to about 7.5.

13. The isolated protein isoform of claim 11 wherein the one or more isoforms are selected from the group consisting of: Kininogen isoform with a pl of about 5.6; Kininogen isoform with a pl of about 5.6; Apolipoprotein A1 isoform with a pl of about 5.2; Apolipoprotein A1 isoform with a pl of about 5.3; Apolipoprotein A1 isoform with a pl of about 5.4; Apolipoprotein A1 isoform with a pl of about 5.6; Apolipoprotein A1 isoform with a pl of about 5.7; Apolipoprotein A1 isoform with a pl of about 5.8; Retinol Binding Protein 4 isoform with a pl of about 5.1; Retinol Binding Protein 4 isoform with a pl of about 5.5; Retinol Binding Protein 4 isoform with a pl of about 5.9; Retinol Binding Protein 4 isoform with a pl of about 6.6; Transthyretin isoforms with a pl of about 5.1; Transthyretin isoforms with a pl of about 5.9; Transthyretin isoforms with a pl of about 6.3; Transthyretin isoforms with a pl of about 6.8; Transthyretin isoforms with a pl of about 7.1; and Transthyretin isoforms with a pl of about 7.3.

14. The isolated protein isoform of claim 11 wherein the protein isoform is substantially purified.

15. The isolated protein isoform of claim 11 wherein the protein isoform is a human protein isoform or a mouse protein isoform.

16. A method for isolating a protein isoform selected from the group consisting of: Kininogen isoforms, Apolipoprotein A1 isoforms, Retinol Binding Protein 4 isoforms, Haptoglobin isoforms, and Transthyretin isoforms comprising isolating the protein isoform using gel electrophoresis, HPLC, or LC.

17. The method of claim 16 wherein the one or more protein isoforms are selected from the group consisting of: Kininogen isoform with a pl of about 5.6; Kininogen isoform with a pl of about 5.6; Apolipoprotein A1 isoform with a pl of about 5.2; Apolipoprotein A1 isoform with a pl of about 5.3; Apolipoprotein A1 isoform with a pl of about 5.4; Apolipoprotein A1 isoform with a pl of about 5.6; Apolipoprotein A1 isoform with a pl of about 5.7; Apolipoprotein A1 isoform with a pl of about 5.8; Retinol Binding Protein 4 isoform with a pl of about 5.1; Retinol Binding Protein 4 isoform with a pl of about 5.5; Retinol Binding Protein 4 isoform with a pl of about 5.9; Retinol Binding Protein 4 isoform with a pl of about 6.6; Transthyretin isoforms with a pl of about 5.1; Transthyretin isoforms with a pl of about 5.9; Transthyretin isoforms with a pl of about 6.3; Transthyretin isoforms with a pl of about 6.8; Transthyretin isoforms with a pl of about 7.1; and Transthyretin isoforms with a pl of about 7.3.

18. The method of claim 16 wherein the method comprises excising a spot from the electrophoretic gel.

19. The method of claim 16 wherein the method comprising excising a spot from an electrophoretic gel
wherein the electrophoretic gel is a 2D gel where one of the dimensions is a separation by isoelectric focusing or is a Western blot.

20. The method of claim 16 wherein the protein isoform is a human protein isoform or a mouse protein isoform.

21. A kit for the diagnosis of a stage of diabetes in a mammal comprising a composition used for the detection of a protein isoform selected from the group consisting of: Kininogen isoforms, Apolipoprotein A1 isoforms, Retinol Binding Protein 4 isoforms, Haptoglobin isoforms, and Transthyretin isoforms.

22. The kit of claim 21 wherein the composition comprises a polyclonal or monoclonal antibody designed to detect the protein isoform.

23. The kit of claim 21 wherein the mammal is a human or a mouse.

24. The method of claim 1 wherein the analyzing comprises comparing one or more protein isoform concentrations to a normal state baseline of one or more protein isoform concentrations.

25. The method of claim 1 wherein the method further comprises the step of determining a normal state baseline of one or more protein isoform concentrations, prior to the measuring.
